(19) United States
(12) Patent Application Publication
(10) Pub. No.: US 2011/0064828 A1
(43) Pub. Date: Mar. 17, 2011

(54) TREATMENT OF METASTATIC TUMORS AND OTHER CONDITIONS

(75) Inventors: Chris Pazoles, Westborough, MA (US); Evgeny Vulfson, Irvington, NY (US); Qihong Huang, Philadelphia, PA (US)

(73) Assignees: Novelos Therapeutics, Incorporated, Newton, MA (US); The Wistar Institute, Philadelphia, PA (US)

(21) Appl. No.: 12/879,073

(22) Filed: Sep. 10, 2010

Related U.S. Application Data
(60) Provisional application No. 61/241,808, filed on Sep. 11, 2009.

(51) Int. Cl. A61K 33/24; A61K 38/06; A61P 35/00

(52) U.S. Cl. 424/649; 514/19.8

(57) ABSTRACT

The present invention generally relates to pharmacology and, in particular, to the treatment of tumors and other conditions. In some aspects, the invention is directed to the treatment of subjects having tumors or cancers that are metastatic. Surprisingly, it has been found that certain compositions comprising oxidized glutathione-based compounds are able to effectively treat such cancers by inhibiting cell migration and/or invasion processes, and thus, inhibiting tumor cell metastases. Without being bound by any theory, it is believed that such compositions are effective since the compositions are able to suppress the activation of critical signaling pathways within cells that are used for cell migration, such as the ErbB2 and/or phosphoinositide-3 kinase (PI3K) pathways, including the downstream RhoA and AKT pathways. Such pathways are regulated by ERp5, which is a protein disulfide isomerase regulated using certain redox pathways, and those redox pathways are unusually sensitive to treatment using oxidized glutathione-based compounds. Thus, the composition of the instant invention, in some embodiments, are surprisingly effective at preventing tumor metastases. While other references have disclosed the treatment of cancers using oxidized glutathione-based compounds, no reference has suggested that signaling pathways used for cell migration, invasion and metastasis, such as the ErbB2, PI3K, RhoA, and AKT pathways, are highly susceptible to treatment by altering the redox state of the cell, e.g., by oxidized glutathione-based compounds. Accordingly, the use of such compositions to treat tumor metastases is surprising and could not be predicted given the teachings of the prior art.

### RELATED APPLICATIONS

This application claims the benefit of U.S. Provisional Patent Application Ser. No. 61/241,808, filed Sep. 11, 2009, entitled “Treatment of Metastatic Tumors and Other Conditions,” by Pazoles, et al., incorporated herein by reference.

### FIELD OF INVENTION

The present invention generally relates to pharmacology and, in particular, to the treatment of metastatic tumors and other conditions.

### BACKGROUND

GSSG is known as a dimer of tripeptide glutathione (gamma-glutamyl-cysteinyl-glycine) where two molecules of the tripeptide with the above structure are linked via a covalent disulfide bond between the cysteine residues. Both the tripeptide glutathione (glutathione, reduced glutathione, GSH; hereinafter referred to as GSH) and its dimer GSSG (oxidized glutathione) are endogenous substances present in animal tissues and biological fluids. The ratio of GSSG to GSH (GSSG:GSH) is a primary determinant of redox status in cells, tissues and biological fluids. Under natural conditions, changes in GSSG:GSH (i.e. in redox status) regulate cell functions via protein S-glutathionylation, a post-translational structural modification of proteins involving covalent interaction of the cysteine thiol of GSH with reactive thiol groups in the protein. This structural alteration is known to modify protein function (inhibition in some cases, activation in others). Proteins subject to regulation by S-glutathionylation include enzymes, receptors, ion channels and other functional protein classes.

GSSG is known to be used as a component of a nutritional supplement utilized as an adjunct diet in treating patients. However, being a peptide substance composed of amino acids, most of the orally administered GSSG is digested in the gastrointestinal tract and does not enter the general circulation. As a result, oral delivery of GSSG fails to influence physiological processes regulated by GSSG:GSH.

Therefore, administration of GSSG via other routes (for example by intravenous and/or subcutaneous injection) is necessary to deliver GSSG to target cells and tissues in order to alter GSSG:GSH and modify cell or tissue redox status and, as a consequence, biological processes that are linked to redox status such as cell signaling pathway activity leading to activation or inhibition of processes such as endogenous production of cytokines and hematopoietic factors, regulation of cytoskeletal architecture, apoptosis and cell proliferation.

### SUMMARY OF THE INVENTION

The present invention generally relates to pharmacology and, in particular, to the treatment of metastatic tumors and other conditions. The subject matter of the present invention involves, in some cases, interrelated products, alternative solutions to a particular problem, and/or a plurality of different uses of one or more systems and/or articles.

Several method are disclosed herein of administering a compound to a subject for prevention or treatment of a particular condition. It is to be understood that in each such aspect of the invention, the invention specifically includes, also, the compound for use in the treatment or prevention of that particular condition, as well as use of the compound for the manufacture of a medicament for the treatment or prevention of that particular condition.

In one aspect, the invention is generally directed to a method of inhibiting metastasis of tumor cells. In one set of embodiments, the method includes an act of administering, to a subject diagnosed as having a metastatic tumor, a composition comprising an oxidized glutathione-based compound. In some cases, the compound may inhibit metastasis of the tumor cells.

In another aspect, the invention is generally directed to the use of a composition in the preparation of a medicament for treatment of a metastatic tumor. In one set of embodiments, the composition comprises a compound comprising an oxidized glutathione-based compound

In another aspect, the present invention is directed to a method of making one or more of the embodiments described herein. In another aspect, the present invention is directed to a method of using one or more of the embodiments described herein.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each to embodiment of the invention shown where illustration is not necessary to allow those of ordinary skill in the art to understand the invention. In the figures:

FIG. 1 illustrates a cell migration/invasion chamber, in accordance with one embodiment of the invention;

FIGS. 2A-2C illustrate results from a tumor cell invasion assay, in another embodiment of the invention;

FIGS. 3A-3C illustrate results from a tumor cell migration assay, in yet another embodiment of the invention;

FIG. 4 illustrates results from a tumor cell cytotoxicity assay, with a composition of the invention;

FIG. 5 illustrates a model of signaling pathways regulating tumor cell migration, invasion, and metastasis, in another embodiment of the invention;

FIG. 6 illustrates levels of activated (phosphorylated) ErbB2 and PI3K, in accordance with another embodiment of the invention;

FIG. 7 shows an example synthesis scheme of a composition of the oxidized glutathione disodium salt with cis-diamminedichloroplatinum;

FIG. 8 illustrates results expression of phosphorylated ErbB2 and PI3K, in accordance with one embodiment of the invention; and

FIG. 9 illustrates expression of Akt and RhoA, in accordance with another embodiment of the invention.

### DETAILED DESCRIPTION

The present invention generally relates to pharmacology and, in particular, to the treatment of metastatic tumors and other conditions. In some aspects, the invention is directed to the treatment of subjects having tumors or cancers that are metastatic. Compositions comprising oxidized glutathione-based compounds, such as the compositions exemplified below, are known to effectively treat certain cancers, a property believed to be related to their ability to increase hematopoiesis, immune stimulation and chemosensitivity of tumor cells. Surprisingly, it has been found that certain compositions comprising oxidized glutathione-based compounds are also able to effectively treat such cancers by inhibiting cell migration and/or invasion processes, and thus, inhibiting tumor cell metastases. Without being bound by any theory, it is believed that such compositions are effective since the compositions are able to suppress the to activation of critical signaling pathways within cells that are used for cell migration and/or invasion, such as the ErbB2 and/or phosphoinositide-3 kinase (PI3K) pathways, including the downstream RhoA and AKT pathways. Such pathways are regulated by ERp5, which is a protein disulfide isomerase regulated using certain redox pathways, and those redox pathways are unusually sensitive to treatment using oxidized glutathione-based compounds. Thus, the composition of the instant invention, in some embodiments, are surprisingly effective at preventing tumor metastases. While other references have disclosed the treatment of cancers using oxidized glutathione-based compounds, no reference has suggested that signaling pathways used for cell migration and/or invasion, such as the ErbB2 and PI3K pathways (including RhoA and AKT), are highly susceptible to treatment by altering the redox state of the cell, e.g., by oxidized glutathione-based compounds, and none have suggested such compounds could inhibit cell migration and invasion. Accordingly, the use of such compositions to treat tumor metastases is surprising and could not be predicted given the teachings of the prior art.

In one aspect, the present invention is generally directed to treatment of subjects diagnosed as having or at risk for a metastatic tumor. In certain embodiments, as discussed below, a composition comprising a compound comprising an oxidized glutathione-based compound is given to a subject. The composition, in some cases, may include an extender (for example, inosine), and/or a metal material, for instance, comprising platinum or palladium (e.g., cis-platin).

A metastatic tumor is generally a tumor or cancer in which the cancer cells forming the tumor have a high potential to, or have begun to, metastasize, or spread from one location to another location or locations within a subject, for example, creating secondary tumors within the subject. Such metastatic behavior may be indicative of malignant tumors. In some cases, as discussed below, metastatic behavior may be associated with an increase in cell migration and/or invasion behavior of the tumor cells.

In one set of embodiments, the metastatic potential of a tumor can be identified by culturing the tumor cells and determining their migration and/or invasion activity, e.g., using a Matrigel™ invasion assay or similar assays. In one set of embodiments, the transwell Matrigel™ invasion assay may be performed using 100 microliters of cells at a density of 10⁶ cells/ml in a donor chamber and 600 microliters of suitable cell culture media appropriate for the cells in a receiving chamber separated by a membrane through to which cells can migrate, e.g., in a 24-well microtiter plate. To assess cell invasion, a layer of 1 mg/ml Matrigel™ having a thickness of about 100 mm is added to the upper surface of the membrane. This represents the extracellular matrix through which invading cells need to penetrate to effect metastasis. After incubation at 37° C. for 24 hours, the number of cells that have migrated from the donor chamber to the receiving chamber can be counted or estimated. A migrating and/or invading tumor may be characterized as exhibiting the migration and/or invasion of at least about 500 migrating and/or invading cells, at least about 1000 migrating and/or invading cells, at least about 1500 migrating cells, at least about 2000 migrating and/or invading cells, or at least about 3000 migrating and/or invading cells under such conditions.

Non-limiting examples of tumors that can be treated include non-small cell lung cancer, breast cancer, ovarian cancer, or colorectal cancer. Other examples of cancers include biliary tract cancer; bladder cancer; brain cancer including glioblastomas and medulloblastomas; breast cancer; cervical cancer; choriocarcinoma; colon cancer; endometrial cancer; esophageal cancer; gastric cancer; hematological neoplasms including acute lymphocytic and myelogenous leukemia; multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Kaposi's sarcoma, basocellular cancer, and squamous cell cancer; testicular cancer including germinal tumors such as seminoma, non-seminoma, teratomas, choriocarcinomas; stromal tumors and germ cell tumors; thyroid cancer including thyroid adenocarcinoma and medullar carcinoma; and renal cancer including adenocarcinoma and Wilms' tumor. Commonly encountered cancers include breast, prostate, lung, ovarian, colorectal, and brain cancer.

Surprisingly, it has been found that the compositions described herein are able to suppress the activation of critical signaling pathways within cells that are used for cell migration and/or invasion, in particular, the ErbB2 and phosphoinositide-3 kinase (PI3K) pathways, including RhoA and AKT. Such pathways are regulated by ERp5, which is a protein disulfide isomerase that is regulated using certain redox pathways, and those redox pathways are unusually sensitive to treatment using oxidized glutathione-based compounds. Accordingly, by limiting the ability of the tumor cells to migrate and/or invade, by affecting these pathways, the ability of the tumor cells to metastasize is greatly reduced. Thus, by treating cells with compositions comprising an oxidized glutathione-based compound, which alters the redox potential of the cells, the ability of tumors cells to metastasize is unexpectedly reduced.

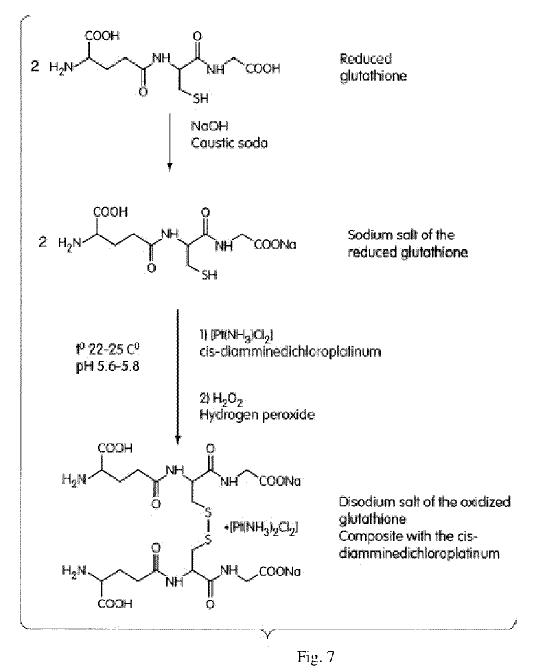

As mentioned, certain embodiments of the present invention are generally directed to a composition comprising a compound comprising an oxidized glutathione-based compound. An example of an oxidized glutathione-based compound is oxidized glutathione; other examples are discussed below. Oxidized glutathione (also known as glutathione disulfide and GSSG) will often be referred to as “GSSG” in this application. An “oxidized glutathione-based compound” refers to any compound having a basic dimer structure where each unit of the dimer comprises a glutamic acid group or salt or derivative bonded to a cysteine group or salt or derivative bonded to a glycine group or salt or derivative, and each unit is correspondingly bonded to each other by the cysteine sulfur atoms to form a sulfur-sulfur bond (disulfide bond). Derivatives of these, which are also included as oxidized-glutathione based compounds, can be prepared by reacting at least one reactive group of these compounds (or precursors thereof) with another chemical species. As mentioned, an example of an oxidized glutathione-based compound is GSSG itself; an example synthesis scheme can be found in FIG. 7.

Accordingly, in one embodiment, the oxidized glutathione-based compound has the general formula:

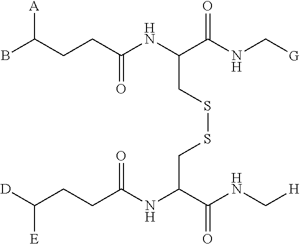

In this formula, A, B, D, E, G and H can each be selected from the group consisting of an organic unit and salts of the organic unit. In some instances, the “organic unit” may be chosen to allow the oxidized glutathione-based compound to remain soluble in biological media. In certain embodiments, the organic unit may be chosen to not impart toxicity to the oxidized glutathione-based compound in an applied dosage. It should be understood to that A, B, D, E, G and H can independently be the same or different. In some embodiments, groups A-H can each include a unit selected from the group consisting of amine groups, carboxyl groups, and amides. For example, A-H can represent amino acids or derivatives bonded via an amide bond. Alternatively, any two of A-H can be linked to each other by at least one covalent bond. Thus, A-H can be part of a cyclic structure.

One aspect of the present invention provides a composition comprising an oxidized glutathione-based compound and a metal material. Generally, a “composition” can include one or a mixture of different chemical species. The “mixture” can be a physical mixture or a chemical mixture, i.e., having a chemical interaction involving either a chemical bond or an electrostatic interaction. In one embodiment, the mixture can be prepared by dissolving and/or suspending different chemical species in a solution or other liquid and precipitating out or filtering out a resulting solid. In another embodiment, the mixture can be a homogeneous solution comprising the different chemical species.

In one embodiment, the composition comprises an excess of the oxidized glutathione-based compound relative to the metal material. Non-limiting examples of such an excess are in a ratio of between about 3000:1 and about 1:1, in a ratio of between about 1000:1 and about 1:1, in a ratio of between about 1500:1 and about 500:1, in a ratio of between about 1200:1 and about 800:1, in a ratio of between about 3000:1 and about 500:1, in a ratio of between about 1000:1 and about 10:1, or in a ratio of between about 1000:1 and about 100:1, etc. In another embodiment, the composition comprises equal amounts of the oxidized glutathione-based compound and the metal material, i.e., a ratio of about 1:1. In one embodiment, the composition has a ratio of the oxidized glutathione-based compound and the metal material of 1000:1. In one embodiment, the composition has a ratio of the oxidized glutathione-based compound and the metal material of 10,000:1.

In one embodiment, the metal material comprises a metal selected from the group consisting of platinum and palladium. One or both metals may be present in various embodiments (and in some cases, additional metals may be present as well). In some (but not all) embodiments, small portions of the metal material can be insoluble, as long as the insoluble portion does not result in any toxic or hazardous effects to the biological system. If a platinum material is present, the platinum material can be selected from the group consisting of a platinum salt, a coordination compound and an organometallic compound. For example, the platinum material may be a platinum coordination compound such as cis-platin (cis-Pt(NH₃)₂Cl₂ or cis-diamminedichloroplatinum).

In one set of embodiments, the oxidized glutathione-based compound may be present as oxidized glutathione itself (GSSG) and/or salts thereof, where both A and E are —CO₂H, both B and D are —NH₂ and both G and H are —CO₂M, M being a counterion. The counterion can be any suitable counterion, e.g., a proton (H⁺), an organic-based ion such as tetralkyl-amononium, an alkaline metal (e.g., Na⁺, K⁺, Li⁺), an alkaline earth metal (e.g., Mg²⁺, Ca²⁺), or a transition metal (e.g., zinc, molybdenum, vanadium, iron, or the like). Other counterions are also possible. The counterion may or may not have a biological effect; for example, the counterion may be selected on the basis of charge balance, ease of manufacture, biological inertness, solubility, and/or other suitable criteria. It should be understood that in aqueous media, any of A-H can, in some (but not all) embodiments, independently comprise an ionized group, e.g., A and E can be —CO₂⁻, and B and D can be —NH₂⁺ and the ionized groups are neutralized by an appropriate counterion, such as those discussed above. Additional examples of oxidized glutathione-based compounds include GSSG, and those disclosed in U.S. Pat. No. 6,312,734, incorporated herein by reference in its entirety. Still other examples of oxidized glutathione-based compounds are disclosed in U.S. Pat. No. 6,165,979; U.S. Pat. No. 6,251,857; U.S. Pat. No. 6,492,329; U.S. Pat. No. 7,169,412; U.S. Pat. No. 7,371,411; Int. Pat. Apl. Pub. No. WO 97/21443; Int. Pat. Apl. Pub. No. WO 97/21444; Int. Pat. Apl. Pub. No. WO 00/31120; and Int. Pat. Apl. Pub. No. WO 97/21444, each of which is incorporated herein by reference in its entirety.

In one set of embodiments, the composition comprising an oxidized glutathione-based compound may include a GSSG derivative. The glutathione can be derivatized after preparation of the composition, or it can be derivatized prior to preparation of the composition, i.e., the GSH can be derivatized prior to oxidation. As examples of various derivatized compounds suitable for use in various embodiments of the present invention, see U.S. Pat. No. 6,312,734, incorporated herein by reference in its entirety.

For example, the GSSG derivative may, in some embodiments, be selected from the group of compounds representing a molecule of GSSG chemically modified by binding covalently as for example: with cysteamine-(2-mercaptoethylamine), lipoic acid (6,8-thioctic acid), carnosine (beta-alanyl-hystidine), adenosine (9-beta-D-ribofuranosyladenine), methionine (2-amino-4-[methylthio]butanoic acid); and both the D and L forms of the amino acids set forth in this paragraph can be used in certain cases. In some embodiments, the derivatives of GSSG may be covalently bound either to cysteamine (S-thioethylamine-glutathione disulfide), or to lipoic acid (bis-[6,8-thiooktanil]glutathione disulfide), or to carnosine ([beta-alanyl-hystidil]glutathione disulfide), or to adenosine ([9-beta-D-ribofuranosyladenil]glutathione disulfide), or to methionine (bis-[2-amino-4-[methylthio]butanoil]glutathione disulfide), or mixtures thereof and including the D and/or L forms of amino acids herein.

Other derivatives include, but are not limited to, derivatives in the form of sodium, lithium, potassium, calcium, zinc, molybdenum, vanadium and other salts of GSSG and/or the above-described compounds, as well as derivatives obtained through covalent binding to phenylalanine, or to methionine and some other amino acids including D and L forms of the amino acids herein; or to cystamine, lipoic acid, or to inosine. Further non-limiting examples of derivatives include compounds in which GSSG is covalently bound to cysteamine (S-thioethylamine-glutathione disulfide), lipoic acid (bis-[6,8-thiooktanil]glutathione disulfide), carnosine ([b-alanyl-hystidil]glutathione disulfide), adenosine ([9-beta-D-ribofuranosyladenil]glutathione disulfide), or methionine (bis-[2-amino-4-[methylthio]butanoil]glutathione disulfide). Additional examples of derivatives include replacement of one or more of L-amino-acids constituting the molecule of both GSSG and the aforementioned derivatives with their D-forms.

In one set of embodiments, an extender may be used in the composition. As used herein, the term “extenders” includes any compound able to substantially extend the lifetime of the oxidized glutathione-based compound in the body, relative to the lifetime of the oxidized glutathione-based compound in the body in the absence of the extender. For example, the extender may have oxidant activity, be able of forming weak ionic and/or coordinating links which stabilize the oxidized glutathione-based compound, be a competitor of NADP-H-dependent reduction of the oxidized glutathione-based compound as catalyzed by glutathione reductase, or be able to produce inhibition of reduction of NADP⁺ into NADP-H catalyzed by glucose-6-phosphate-dehydrogenase or by other NADP-H-dependent enzymes. Glucose-6-phosphate-dehydrogenase is an enzyme of the pentose phosphate pathway that may react with the oxidized glutathione-based compound.

Non-limiting examples of extenders include hydrogen peroxide (e.g., at 0.003%), ascorbic acid (e.g., at 5.0%), or other compounds with oxidant activity; dimethyl sulfoxide (e.g., at 7.0%), or other compounds capable of forming weak ionic and/or coordinating links which are able to stabilize oxidized glutathione-based compounds; inosine (hypoxanthine-9-D-ribofuranoside) (e.g., at 0.1%), or its derivatives, including inosine nucleosides; or cystamine (2,2′-Ditio-bis[ethylamine] (e.g., at 0.1%) or other compounds able to produce reversible inhibition of glucose-6-phosphate-dehydrogenase. Specific examples of extenders include, but are not limited to, hydrogen peroxide, inosine, ascorbic acid, dimethyl sulfoxide, or cystamine or mixtures thereof.

In some embodiments, the extender may include pharmaceutically acceptable pro-oxidant compounds (e.g., hydrogen peroxide, ascorbic acid), compounds capable of forming both weak ionic and coordinating links which can stabilize the oxidized glutathione-based compound (e.g., dimethyl sulfoxide). For instance, an oxidant such as hydrogen peroxide or ascorbic acid is a donor of reactive oxygen intermediates may cause the oxidized glutathione-based compound to be reduced by glutathione reductase at a lesser speed, thereby causing a slower reduction of the oxidized glutathione-based compound. If hydrogen peroxide is used, it can be present, in certain cases, in amounts of from about 0.03 to about 0.0003% by weight of solutions used (from 1.0 to 5.0 ml of solutions, regardless whether they contain or do not contain an oxidized glutathione-based compound. Ascorbic acid can be used in certain embodiments, if present, in amounts of from 0.1 to 10% by weight of solutions used (from 1.0 to 10.0 ml of solution).

Other pharmaceutically acceptable extenders may be used in some cases. A non-limiting example of a compound able to form weak ionic and/or coordinating links which stabilize molecules of oxidized glutathione-based compound is dimethyl sulfoxide. Examples of compounds able to competitively react with a reduced form of the nicotinamide adenine dinucleotide phosphate or NADP-H are inosine and other derivatives of hypoxanthine). Non-limiting examples of compounds able to reversibly inhibit the processes of reduction of the oxidized form of NADP into NADPH include cystamine (2,2′-Dithio-bis[ethylamine]) and other inhibitors of glucose-6-phosphate-dehydrogenase. For example, dimethyl sulfoxide may be present as 7.0% (v/v) solution, or as a solution of from 0.1% to 30% by volume (e.g., from 1.0 to 30.0 ml of solutions or more when applied epicutaneously/through instillations).

Without being bound by any theory, it is believed that since reduced NADP-His a cofactor of the glutathione reductase system which is able to catalyze the reduction of certain oxidized glutathione-based compounds, any pharmaceutically acceptable compounds or biophysical influence retarding this reduction or blocking biological oxidation of NADP-H by glutathione reductase will facilitate preservation of the oxidized glutathione-based compound from reduction and, therefore, may enhance and prolong its curative effect.

Another example of an extender is inosine (hypoxanthine-9-D-ribofuranoside). Inosine may be present, for example, as 0.1% solution, from 0.1% to 5% by weight (e.g., from 1.0 to 5.0 ml of solution), or in a molar ratio of 1:1 (oxidized glutathione-based compound:inosine), or in any other suitable molar ratio (e.g., a ratio of between about 3000:1 and about 1:1, in a ratio of between about 1000:1 and about 1:1, in a ratio of between about 1500:1 and about 500:1, in a ratio of between about 1200:1 and about 800:1, in a ratio of between about 3000:1 and about 500:1, in a ratio of between about 1000:1 and about 10:1, or in a ratio of between about 1000:1 and about 100:1, etc). Without being bound by any theory, inosine may have the ability to compete with NADP-H, and thereby, to retard reduction of oxidized glutathione-based compounds. Similarly, other hypoxanthine derivatives (including inosine, nucleoside ones, hypoxanthine riboside and other nucleoside derivatives of inosine) may possess this property as well.

Yet another example of an extender is cystamine (2,2′-dithio-bis[ethylamine]). It may be present as 0.1% solution, or a solution of from 0.1% to 3% by weight (for example, 1.0 to 5.0 ml of solution)

In some embodiments, an enhancer/modulator may be used in the composition. As used herein, the enhancer/modulator includes a material which increases or changes beneficially in terms of curative outcomes the therapeutic effect of the oxidized glutathione-based compound, but is not an extender of half life of the oxidized glutathione-based compound. Non-limiting examples of enhancer/modulators include methyl moiety donators (such as choline chloride{[2-hydroxyethyl]trimethylammonium chloride} or S-adenosyl-methionine), representatives of intracellular redox-oxidative pairs (such as lipoic/dehydrolipoic, folic/dehydrofolic, ascorbic/dehydroascorbic acids). Specific non-limiting examples include choline chloride, S-adenosyl-methionine, lipoic (6,8-thioctic) and folic (pteroylglutamic) acids. For instance, the enhancer/modulator may include choline chloride (e.g., 10%), S-adenosyl-methionine (e.g., 5.0%), or other pharmaceutically acceptable donators of methyl groups; lipoic acid (e.g., 0.5%); or folic acid (e.g., 0.5%) or other compounds, which are capable of formation intracellular redox-oxidative pairs.

Donators of methyl groups, such as choline chloride and S-adenosyl-methionine used in combination with oxidized glutathione-based compound have appeared to be more effective compared with oxidized glutathione-based compound alone when these agents are used for treating animals with experimental pathologic conditions of immunologic and infectious nature. It has been shown that choline-chlorine can be used in subjects as 10% solution, or as a solution of from 1.0% to 20% by weight (from 1.0 to 5.0 ml or solution). S-adenosyl-methionine can be used in subjects, e.g., as a 5.0% solution, or as a solution of from 1.0% to 10% by weight (from 1.0 to 5.0 ml of solution).

Compounds which are capable of formation intracellular redox-oxidative pairs (lipoic, folic and ascorbic acids) have also been found to augment effects of the oxidized glutathione-based compound in immunologic, infectious, or other diseases (diabetes mellitus). Lipoic acid can be used in subjects, e.g., as a 0.5% solution, or as a solution of from 0.1% to 1.0% by weight (from 1.0 to 5.0 ml of solution). Folic acid can be used in subjects, e.g., as a 0.5% solution, or as a solution of from 0.1% to 1.0% by weight (from 2.0 to 5.0 ml of solution).

An oxidized glutathione-based compound can either be administered combined in a single dosage form with an extenders and/or an enhancer/modulator, or can be delivered into a subject separately from either or both of the extender and/or the enhancer/modulator, using different suitable pharmaceutically acceptable administration routes for each constituent of any combination used.

The compositions described herein can be prepared by various methods, according to various aspects. For example, in one embodiment, a composition may be prepared from the addition of a metal material to glutathione in the presence of an oxidant to produce an oxidized glutathione. In another embodiment, the composition can be prepared by the addition of a metal material to an oxidized glutathione-based compound. In addition, an example synthesis scheme can be found in FIG. 7. Many oxidized glutathione-based compounds, including oxidized glutathione, are available commercially, and many metal materials, such as cis-platin, can be obtained commercially. Many extenders (e.g., inosine) or enhancer/modulators (e.g., choline chloride) are also available commercially. Further, techniques for producing certain compositions comprising an oxidized glutathione-based compounds can be found in U.S. Pat. No. 6,165,979; U.S. Pat. No. 6,251,857; U.S. Pat. No. 6,492,329; U.S. Pat. No. 6,312,734; U.S. Pat. No. 7,169,412; U.S. Pat. No. 7,371,411; Int. Pat. Apl. Pub. No. WO 97/21443; Int. Pat. Apl. Pub. No. WO 97/21444; Int. Pat. Apl. Pub. No. WO 00/31120; or Int. Pat. Apl. Pub. No. WO 97/21444, each incorporated herein by reference in its entirety.

Certain aspects of the invention provides for the stabilization of disulfide bonds of an oxidized glutathione-based compound. “Stabilizing a disulfide bond,” as used herein, refers to a process for maintaining a bond between two sulfur atoms and preventing facile reversion of the oxidized glutathione-based compound (e.g., GSSG) back to the reduced form (e.g., GSH). In some cases, stabilizing the disulfide bond can result in an increased lifetime of the oxidized glutathione-based compound in the body. In the presence of certain reductants, the disulfide bond can cleave resulting in formation of the reduced form of the glutathione-based compound, which is an undesired reaction. By maintaining the glutathione-based compound in an oxidized form for a greater amount of time, the compound can be pharmaceutically effective for a correspondingly longer period of time in biological media.

In one set of embodiments, the disulfide bond can be stabilized by interacting the oxidized glutathione-based compound with a metal material. The metal material may be a platinum- or palladium-containing material, such as cis-platin. In some cases, the platinum material is present in an amount of between about 0.0003 molar equivalent to about 1 molar equivalent relevant to the oxidized glutathione-based compound, or in a ratio of between about 0.001 molar equivalent to about 1 molar equivalent relevant to the oxidized glutathione-based compound. Other ratios include those described above, e.g., a ratio of the oxidized glutathione-based compound to the metal material of between about 3000:1 and about 1:1, in a ratio of between about 1000:1 and about 1:1, in a ratio of between about 1500:1 and about 500:1, in a ratio of between about 1200:1 and about 800:1, in a ratio of between about 3000:1 and about 500:1, in a ratio of between about 1000:1 and about 10:1, or in a ratio of between about 1000:1 and about 100:1, etc.

In one embodiment, “interacting the oxidized glutathione-based compound with a metal material” comprises providing a glutathione-based compound and reacting this compound with an oxidant and a metal material, such as a platinum material. A “glutathione-based compound” refers to any compound having a structure comprising a glutamic acid/salt/derivative bonded to a cysteine/salt/derivative bonded to a glycine/salt/derivative. Non-limiting examples of glutathione-based compound include glutathione itself or any derivative, where a derivative can be prepared by reacting a reactive group with another chemical species. The resulting product will be an oxidized glutathione-based compound having a stabilized disulfide bond. Thus, in some embodiments, a glutathione-based compound is in a reduced form, such as GSH, and the reaction with a oxidant involves oxidizing the glutathione-based compound to produce a sulfur-sulfur bond. The oxidant can be any species which can cleave a S—H bond of a glutathione-based compound to produce a hydrogen atom and a compound having a sulfur-based radical which ultimately can react with another sulfur-based radical to provide the sulfur-sulfur bond. Various oxidants that can perform this S—H bond cleavage are well known in the art. In certain embodiments, the oxidant includes oxygen and/or hydrogen peroxide. Other oxidants can also be used in certain cases.

In some cases, reacting a glutathione-based compound with an oxidant and the metal material comprises an oxidation reaction. Relative amounts of the reactants may include about 1 molar equivalent of the glutathione-based compound with less than about to 1 molar equivalent of the oxidant such as hydrogen peroxide, or with about 0.9 molar equivalent of the hydrogen peroxide. In some embodiments, the oxidation reaction comprises reacting about 1 molar equivalent of the glutathione-based compound with between about 0.0003 molar equivalent and about 1 molar equivalent of the platinum material, between about 0.001 molar equivalent and about 1 molar equivalent of platinum material, between about 0.001 molar equivalent and about 0.1 molar equivalent, or between about 0.001 molar equivalent and 0.01 molar equivalent, in the presence of less than 1 molar equivalent of the oxidant. In other embodiments, about 1 molar equivalent of the glutathione-based compound is reacted with about 1 molar equivalent of the platinum material in the presence of less than 1 molar equivalent of the oxidant.

In one set of embodiments, the method involves oxidizing the glutathione-based compound with about 0.9 molar equivalent of hydrogen peroxide and about 0.001 molar equivalent of cis-platin. One advantageous feature of some embodiments is an increased rate of oxidation of the glutathione-based compound. Another advantageous feature of certain embodiments is that the yield of the resulting composition may be increased to an amount greater than about 98% and this increased yield may be accompanied by an increased purity in some instances. The purification of this composition may be simplified to a significant degree in that liquid chromatography can be performed to obtain a purity of the composition of greater than 99%, which complies with pharmaceutical standards. Thus, certain embodiments of the invention are directed to purities of at least about 90%, at least, about 95%, at least about 97%, at least about 98%, or at least about 99%.

In some embodiments, the composition may be synthesized by oxidizing the reduced glutathione in the presence of cis-diamminedichloroplatinum, which may function as an oxidation reaction catalyst in some cases. The reaction conditions can be regulated in some embodiments by using less than 1 molar equivalent of hydrogen peroxide. Formation of superoxidation products can be reduced, resulting in a near quantitative yield of the product.

In one set of embodiments, the reaction is performed in a solution involving reduced glutathione as a monosodium salt and adding about 0.9 molar equivalent of the hydrogen peroxide and about 0.001 molar equivalent of cis-diamminedichloroplatinum to at room temperature with stirring. The oxidation reaction typically proceeds in about 1.5-2 hours. Control for the completeness of the oxidation process can be conducted by an HPLC assay. The process is completed by the reaction solution lyophilic drying to produce the composition of oxidized glutathione and cis-diamminedichloroplatinum in a mole ratio of 1000:1 (confirmed by spectral analysis on platinum and sodium). The peptide constituent of the obtained composition according to the data of an amino acid assay, a NMR (¹H) spectrum, retention time by HPLC corresponds to GSSG. The admixtures content may not exceed 2%, and the product yield as a disodium salt may be about 96-98% calculating for the dry composition.

While not wishing to be bound by any theory, the increased stabilization of the disulfide bond may be the result of an interaction of the sulfur atoms with the platinum (or other metal) material.

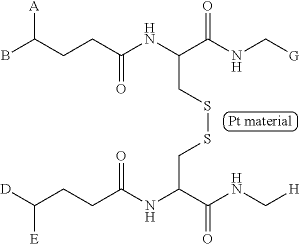

In an interaction between the platinum material and the oxidized glutathione (GSSG) molecule there is a possibility for ligand exchange, i.e., instead of the NH₃ groups, two sulfur atoms possessing two pairs of the lone-pair electrons can be involved in donor/acceptor bonds with the platinum atom. In some cases, the aforesaid stabilization of the disulfide bond may be due to the convergence of the NH₂ groups of the oxidized glutathione-based compound and stabilization of the general GSSG conformation.

It is an advantageous feature of certain embodiments of the present invention that obtaining derivatives of GSSG can produce a compound having different biological/chemical properties and/or activity. Thus, depending on the desired use of a drug comprising a composition including the oxidized glutathione-based compound, it is possible to obtain a particular drug for treatment of a particular disease. In addition, new to chemical modifications of the oxidized glutathione-based compound, such as aminogroup acylation (for instance, bis-phenylalanyl-glutathione, and etc.), can result in a significant decrease in the risk of secondary reactions due to disulfide bond destruction. Reactions such as S-alkylation, oxidation to the corresponding acids, etc., can cause particular hardships in the working process and, in the case given, can be minimized or excluded.

Another aspect of the invention is generally directed to compositions including hexapeptide bis-(gamma-L-glutamyl)-L-cysteinyl-bis-glycine disodium salt and cis-diamminedichloroplatin (cis-platin). In some embodiments, the present invention provides a new class of medicinal substances “thiopoietins” that can be introduced into biological media, resulting in a new level of metabolism and cellular genetic activity.

In another aspect of the invention, any of the compositions described herein can be combined with other cancer treatments, including but not limited to other anti-cancer agents or drugs. The term “cancer treatment” as used herein, may include, but is not limited to, chemotherapy, radiotherapy, adjuvant therapy, surgery, or any combination of these and/or other methods. Aspects of cancer treatment may vary, for instance, depending on the subject being treated. Examples include, but are not limited to, dosages, timing of administration, duration of treatment, etc. One of ordinary skill in the medical arts can determine an appropriate cancer treatment for a subject.

Examples of anti-cancer agents and drugs that can be used in combination with one or more compositions of the invention include, but are not limited to, any one or more of 20-epi-1,25 dihydroxyvitamin D3,4-ipomeanol, 5-ethynyluracil, 9-dihydrotaxol, abiraterone, acivicin, aclarubicin, acodazole hydrochloride, acronine, acylfulvene, adecypenol, adozelesin, aldesleukin, all-tk antagonists, altretamine, ambamustine, ambomycin, ametantrone acetate, amidox, amifostine, aminoglutethimide, aminolevulinic acid, amrubicin, amsacrine, anagrelide, anastrozole, andrographolide, angiogenesis inhibitors, antagonist D, antagonist G, antarelix, anthramycin, anti-dorsalizing morphogenetic protein-1, antiestrogen, antineoplaston, antisense oligonucleotides, aphidicolin glycinate, apoptosis gene modulators, apoptosis regulators, apurinic acid, ARA-CDP-DL-PTBA, arginine deaminase, asparaginase, asperlin, asulacrine, atamestane, atrimustine, axinastatin 1, axinastatin 2, axinastatin 3, azacitidine, azasetron, azatoxin, azatyrosine, azetepa, azotomycin, baccatin III derivatives, balanol, to batimastat, benzochlorins, benzodepa, benzoylstaurosporine, beta lactam derivatives, beta-alethine, betaclamycin B, betulinic acid, BFGF inhibitor, bicalutamide, bisantrene, bisantrene hydrochloride, bisaziridinylspermine, bisnafide, bisnafide dimesylate, bistratene A, bizelesin, bleomycin, bleomycin sulfate, BRC/ABL antagonists, breflate, brequinar sodium, bropirimine, budotitane, busulfan, buthionine sulfoximine, cactinomycin, calcipotriol, calphostin C, calusterone, camptothecin derivatives, canarypox IL-2, capecitabine, caracemide, carbetimer, carboplatin, carboxamide-amino-triazole, carboxyamidotriazole, carest M3, carmustine, carn 700, cartilage derived inhibitor, carubicin hydrochloride, carzelesin, casein kinase inhibitors, castanospermine, cecropin B, cedefingol, cetrorelix, chlorambucil, chlorins, chloroquinoxaline sulfonamide, cicaprost, cirolemycin, cisplatin, cis-porphyrin, cladribine, clomifene analogs, clotrimazole, collismycin A, collismycin B, combretastatin A4, combretastatin analog, conagenin, crambescidin 816, crisnatol, crisnatol mesylate, cryptophycin 8, cryptophycin A derivatives, curacin A, cyclopentanthraquinones, cyclophosphamide, cycloplatam, cypemycin, cytarabine, cytarabine ocfosfate, cytolytic factor, cytostatin, dacarbazine, dacliximab, dactinomycin, daunorubicin hydrochloride, decitabine, dehydrodidemnin B, deslorelin, dexifosfamide, dexormaplatin, dexrazoxane, dexverapamil, dezaguanine, dezaguanine mesylate, diaziquone, didemnin B, didox, diethylnorspermine, dihydro-5-azacytidine, dioxamycin, diphenyl spiromustine, docetaxel, docosanol, dolasetron, doxifluridine, doxorubicin, doxorubicin hydrochloride, droloxifene, droloxifene citrate, dromostanolone propionate, dronabinol, duazomycin, duocarmycin SA, ebselen, ecomustine, edatrexate, edelfosine, edrecolomab, eflornithine, eflornithine hydrochloride, elemene, elsamitrucin, emitefur, enloplatin, enpromate, epipropidine, epirubicin, epirubicin hydrochloride, epristeride, erbulozole, erythrocyte gene therapy vector system, esorubicin hydrochloride, estramustine, estramustine analog, estramustine phosphate sodium, estrogen agonists, estrogen antagonists, etanidazole, etoposide, etoposide phosphate, etoprine, exemestane, fadrozole, fadrozole hydrochloride, fazarabine, fenretinide, filgrastim, finasteride, flavopiridol, flezelastine, floxuridine, fluasterone, fludarabine, fludarabine phosphate, fluorodaunorunicin hydrochloride, fluorouracil, fluorocitabine, forfenimex, formestane, fosquidone, fostriecin, fostriecin sodium, fotemustine, gadolinium texaphyrin, gallium nitrate, galocitabine, ganirelix, gelatinase inhibitors, gemcitabine, gemcitabine hydrochloride, glutathione to inhibitors, hepsulfam, heregulin, hexamethylene bisacetamide, hydroxyurea, hypericin, ibandronic acid, idarubicin, idarubicin hydrochloride, idoxifene, idramantone, ifosfamide, ilmofosine, ilomastat, imidazoacridones, imiquimod, immunostimulant peptides, insulin-like growth factor-1 receptor inhibitor, interferon agonists, interferon alpha-2A, interferon alpha-2B, interferon alpha-N1, interferon alpha-N3, interferon beta-IA, interferon gamma-IB, interferons, interleukins, iobenguane, iododoxorubicin, iproplatin, irinotecan, irinotecan hydrochloride, iroplact, irsogladine, isobengazole, isohomohalicondrin B, itasetron, jasplakinolide, kahalalide F, lamellarin-N triacetate, lanreotide, lanreotide acetate, leinamycin, lenograstim, lentinan sulfate, leptolstatin, letrozole, leukemia inhibiting factor, leukocyte alpha interferon, leuprolide acetate, leuprolide/estrogen/progesterone, leuprorelin, levamisole, liarozole, liarozole hydrochloride, linear polyamine analog, lipophilic disaccharide peptide, lipophilic platinum compounds, lissoclinamide 7, lobaplatin, lombricine, lometrexol, lometrexol sodium, lomustine, lonidamine, losoxantrone, losoxantrone hydrochloride, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, maytansine, mechlorethamine hydrochloride, megestrol acetate, melengestrol acetate, melphalan, menogaril, merbarone, mercaptopurine, meterelin, methioninase, methotrexate, methotrexate sodium, metoclopramide, metoprine, meturedepa, microalgal protein kinase C inhibitors, MIF inhibitor, mifepristone, miltefosine, mirimostim, mismatched double stranded RNA, mitindomide, mitocarcin, mitocromin, mitogillin, mitoguazone, mitolactol, mitomalcin, mitomycin, mitomycin analogs, mitonafide, mitosper, mitotane, mitotoxin fibroblast growth factor-saporin, mitoxantrone, mitoxantrone hydrochloride, mofarotene, molgramostim, monoclonal antibody, human chorionic gonadotrophin, monophosphoryl lipid a/myobacterium cell wall SK, mopidamol, multiple drug resistance gene inhibitor, multiple tumor suppressor 1-based therapy, mustard anticancer agent, mycaperoxide B, mycobacterial cell wall extract, mycophenolic acid, myriaporone, n-acetyldinaline, nafarelin, nagrestip, naloxone/pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, nocodazole, nogalamycin, n-substituted benzamides, O6-benzylguanine, octreotide, okicenone, oligonucleotides, onapristone, ondansetron, oracin, oral cytokine inducer, ormaplatin, osaterone, oxaliplatin, oxaunomycin, oxisuran, paclitaxel, paclitaxel analogs, paclitaxel derivatives, palauamine, palmitoylrhizoxin, pamidronic acid, panaxytriol, panomifene, parabactin, pazelliptine, pegaspargase, peldesine, peliomycin, pentamustine, pentosan polysulfate sodium, pentostatin, pentrozole, peplomycin sulfate, perflubron, perfosfamide, perillyl alcohol, phenazinomycin, phenylacetate, phosphatase inhibitors, picibanil, pilocarpine hydrochloride, pipobroman, piposulfan, pirarubicin, piritrexim, piroxantrone hydrochloride, placetin A, placetin B, plasminogen activator inhibitor, platinum complex, platinum compounds, platinum-triamine complex, plicamycin, plomestane, porfimer sodium, porfiromycin, prednimustine, procarbazine hydrochloride, propyl bis-acridone, prostaglandin J2, prostatic carcinoma antiandrogen, proteasome inhibitors, protein A-based immune modulator, protein kinase C inhibitor, protein tyrosine phosphatase inhibitors, purine nucleoside phosphorylase inhibitors, puromycin, puromycin hydrochloride, purpurins, pyrazofurin, pyrazoloacridine, pyridoxylated hemoglobin polyoxyethylene conjugate, RAF antagonists, raltitrexed, ramosetron, RAS farnesyl protein transferase inhibitors, RAS inhibitors, RAS-GAP inhibitor, retelliptine demethylated, rhenium RE 186 etidronate, rhizoxin, riboprine, ribozymes, RII retinamide, RNAi, rogletimide, rohitukine, romurtide, roquinimex, rubiginone B1, ruboxyl, safingol, safingol hydrochloride, saintopin, sarcnu, sarcophytol A, sargramostim, SDI 1 mimetics, semustine, senescence derived inhibitor 1, sense oligonucleotides, signal transduction inhibitors, signal transduction modulators, simtrazene, single chain antigen binding protein, sizofuran, sobuzoxane, sodium borocaptate, sodium phenylacetate, solverol, somatomedin binding protein, sonermin, sparfosate sodium, sparfosic acid, sparsomycin, spicamycin D, spirogermanium hydrochloride, spiromustine, spiroplatin, splenopentin, spongistatin 1, squalamine, stem cell inhibitor, stem-cell division inhibitors, stipiamide, streptonigrin, streptozocin, stromelysin inhibitors, sulfinosine, sulofenur, superactive vasoactive intestinal peptide antagonist, suradista, suramin, swainsonine, synthetic glycosaminoglycans, talisomycin, tallimustine, tamoxifen methiodide, tauromustine, tazarotene, tecogalan sodium, tegafur, tellurapyrylium, telomerase inhibitors, teloxantrone hydrochloride, temoporfin, temozolomide, teniposide, teroxirone, testolactone, tetrachlorodecaoxide, tetrazomine, thaliblastine, thalidomide, thiamiprine, thiocoraline, thioguanine, thiotepa, thrombopoietin, thrombopoietin mimetic, thymalfasin, thymopoietin receptor agonist, thymotrinan, thyroid stimulating hormone, tiazofurin, tin ethyl etiopurpurin, tirapazamine, titanocene dichloride, topotecan hydrochloride, topsentin, toremifene, toremifene citrate, totipotent stem cell factor, translation inhibitors, trestolone acetate, tretinoin, triacetyluridine, triciribine, triciribine phosphate, trimetrexate, trimetrexate glucuronate, triptorelin, tropisetron, tubulozole hydrochloride, turosteride, tyrosine kinase inhibitors, tyrphostins, UBC inhibitors, ubenimex, uracil mustard, uredepa, urogenital sinus-derived growth inhibitory factor, urokinase receptor antagonists, vapreotide, variolin B, velaresol, veramine, verdins, verteporfin, vinblastine sulfate, vincristine sulfate, vindesine, vindesine sulfate, vinepidine sulfate, vinglycinate sulfate, vinleurosine sulfate, vinorelbine, vinorelbine tartrate, vinrosidine sulfate, vinxaltine, vinzolidine sulfate, vitaxin, vorozole, zanoterone, zeniplatin, zilascorb, zinostatin, zinostatin stimalamer, and zorubicin hydrochloride, as well as salts, homologs, analogs, polymorphs, derivatives, enantiomers, and/or functionally equivalent compositions thereof.

In the applied invention, including examples of the preferred embodiments, the following terminology accepted is used in various aspects.

“Subject in need thereof” as used in this application herein comprises a mammal, e.g., human, domestic animals and livestock including cats, dogs, cattle and horses, which may have one or more manifestations of a disease in which stimulation of endogenous cytokine or hemopoietic factor (or both) production as well as an apoptosis mechanism regulation would be considered beneficial by those skilled in the art with an up-to-date biomedical knowledge.

“Medicinal drug” as used in this application includes any drug form containing any of the compositions of the present invention. The composition may have a therapeutic effect on tumors, especially metastatic tumors and/or other neoplastic diseases, as well as infectious, hematologic, immunologic, neurodegenerative or other diseases.

“Therapeutic effect” indicates any effect in human or other mammals which is beneficial, including curative, preventative, allowing maintenance at a beneficial level, or being in any way advantageous in regard to the subject.

“Pharmaceutically acceptable salt” as used herein comprises any composition that to is in the form of a salt that is acceptable for use in the subject without unwanted detrimental effect on the subject, and including, for example, sodium, lithium, potassium, zinc or vanadium cations, of sodium, potassium lithium, zinc or vanadium salt respectively. As non-limiting examples, the oxidized glutathione-based compound may be a salt, e.g., the sodium salt of GSSG, the lithium salt of GSSG, the potassium salt of GSSG, etc.

“Pharmaceutically acceptable composition” as used herein, may include any suitable composition described herein, or derivative thereof, as a pharmaceutically acceptable substance and may include, in addition, a group of active metabolites or other chemical compounds. For example, the composition can include or can be covalently bound to phenylalanine or to cystamine.

“Metabolism” as used in this application involves the totality of all biochemical reactions taking place within the living organism responsible for vital function maintenance in the said organism.

“Proliferation” as used in this application involves reproduction or multiplication of similar forms (cells) due to constituting (cellular) elements.

“Differentiation” as used in this application involves cell changes including acquisition or possession of features distinguishing from an original with the cell conversion from relatively simple functions to more complex, specialized functions as is in morphological and/or functional heterogeneity incident to the given cellular type through the tissue-specific gene expression.

“Apoptosis” as used in this application involves morphologically distinguishable forms of genetically programmed, physiological cell death initiated by extra- or intracellular signals when there are activated enzymes (e.g., the caspases group) causing destruction (e.g., fragmentation) of nuclear DNA through intranucleosomic cuts and morphologically manifested by (1) cell shrinkage; (2) condensation, margination, and fragmentation of chromatin; and/or (3) retention of cytoplasmic organelle structure, but loss of positional interrelationships; further apoptotic cells or apoptotic bodies formed out of them are engulfed (incur phagocytosis).

“Cytokines” as used herein comprises peptide-origin regulatory compounds produced by the different cell types playing a key role in the immune response development, hemopoiesis and different disease pathogenesis, performing their effect to through gene activation, participating in regulation for all immune system elements (proliferation and differentiation of immune competent cell precursors; antigen representation, antigen-sensitized lymphocyte proliferation, B-lymphocyte differentiation into antibody-producing cells, T-lymphocyte differentiation into functionally different T-lymphocytes; functions of macrophages, neutrophils, eosinophils, mast cells and basophils), as well as controlling growth, differentiation, apoptotic processes and functional activity for different tissue cells (including fibroblasts, chondrocytes, keratinocytes, endotheliocytes, nerve tissue cells and cardiomyocytes).

As used herein, the terms “neoplastic and infectious disease,” “hemopoiesis and immunity depression of various origin,” and “other diseases” mean any neoplastic or infectious disease, any conditions caused or accompanied by the erythroid or myeloid suppression, or a reduction in quantitative or functional immunity parameters, as well as any other disease or pathological condition, in which stimulation/modulation of the aforesaid cytokine and/or hemopoietic factor endogenous production as well as apoptosis mechanism induction would be considered advantageous by those skilled in the art. Thus, modulating the cytokine and hemopoietic factor endogenous production for a person in need thereof by using a composition as described herein. For instance, in one embodiment, the composition may be stabilized using a disulfide bond which, being introduced parenterally, also may influence the cytokine profile allowing regulation of the normal cell metabolism, proliferation, and/or differentiation processes.

Therapeutic effect also includes, in some embodiments, alleviation, prevention or curing of an unwanted body condition and may comprise a process selected from the group consisting of regulating proliferation in normal cells, regulating differentiation in normal cells, and inducing apoptosis of transformed cells where the transformed cells can include diseased cells. The therapeutic effect includes preventative, alleviation and curing effects in various diseases.

“Disease” refers to any unwanted condition of the body including, but not limited to, selected cancers, tumors, and/or other oncological diseases, infectious diseases, immunological diseases, ischemic diseases, neurodegenerative diseases, metabolic diseases, endocrinal diseases, and any other unwanted medical condition.

A “metastatic tumor,” as used herein, is generally a tumor or cancer in which the cancer cells forming the tumor have a high potential to, or have begun to, metastasize, or to spread from one location to another location or locations within a subject, for example, creating secondary tumors within the subject.

In various aspects, the compositions described herein can be administered by any suitable methods, e.g., orally, or as a solution form selected from the group consisting of inhalation solutions, local instillations, eye drops, intranasal introductions, an ointment for epicutaneous applications, intravenous solutions, injection solutions (e.g., subcutaneous, or intravenous), and suppositories. In one set of embodiments, the composition is introduced parenterally or topically. In some cases, the composition may be introduced to enhance the regulatory influence on cell or tissue redox status and/or on signaling pathways controlling for example endogenous production of cytokines and hemopoietic factors, cell proliferation, apopotosis and/or alterations in cytoskeletal architecture in transformed tissues, achieving a corresponding therapeutic effect.

In one embodiment, the composition is administered in a dosage of between about 0.1 mg/kg to about 1.0 mg/kg by body weight of the subject. In another embodiment, the composition is administered in a dosage of between about 1 mg/m² to about 100 mg/m² by body surface of the subject. In another embodiment, the composition can be applied one or more times a day, by one or more day pulses or continuous administration until a desired therapeutic effect is achieved.

In some embodiments, the composition is introduced to the subject at a dose from 0.01 to 1.0 mg of the composition per kg of body weight of the subject for the composition; or at a dose from 1 to 100 mg per 1 m² of body surface of the subject. In certain cases, the composition may be applied epicutaneously/through instillations at a dose from 1 to 100 mg per 1 m² of body surface, for example, at least once during each 24 hour period. Also the composition can be continuously injected or otherwise introduced to the body to have a 24 hour total dosage from 0.01 to 1.0 mg per kg of body weight for the composition, and from 1 to 100 mg per 1 m² of body surface during each 24 hour period. In some instances, administration and introduction of the composition to the subject can be carried out until a desired effect on cell or tissue redox status and/or on signaling pathways controlling for example endogenous production of cytokines and hemopoietic factors, cell proliferation, apoptosis and/or alterations in cytoskeletal architecture in transformed tissues, achieving a corresponding therapeutic effect may be obtained.

Where the composition is administered as a solution, the solution may have, in some embodiments, a concentration of between about 1% to about 10% of the composition. In one set of embodiments, the composition may be, or include, a pharmaceutically acceptable derivative, e.g., for parenteral use is in a pharmaceutically acceptable solvent such as, for example, an aqueous solution including water, glucose solution, isotonic solutions of sodium chloride, buffered salt solutions. Other physiological solvents or carriers can be used in other embodiments. Where the composition is administered as an injectable form, the injectable form may comprise the composition in a solution at any suitable composition, e.g., in a concentration of between about 0.01% to about 3.0%.

For topical application including application for different body cavities, organic solvents or carriers may be used in the form of ointments, pastes, creams or suppositories.

As one non-limiting example, one embodiment of the invention is directed to an injectable solution containing 0.01% to 3% of oxidized glutathione and a platinum material (or a salt thereof) with the dosage range from 0.1 to 1.0 mg/kg by body weight or from 1 to 100 mg/m² of body surface. In cases when the composition is administered like inhalation solutions, local instillations, eye drops, intranasal introduction, or an ointment for epicutaneous applications, or suppositories, an example concentration range is from 1% to 10% of the composition.

Without being bound by any theory, it is believed that a composition including a hexapeptide with the stabilized disulfide bond (e.g., as may be present in an oxidized glutathione-based compound) may be capable of stimulating or beneficially modulating cell or tissue redox status and/or on signaling pathways controlling for example endogenous production of cytokines and hemopoietic factors, cell proliferation, apoptosis and/or cytoskeletal in transformed tissues, achieving a corresponding therapeutic effect. The composition may be applied as a pharmaceutically acceptable formulation in an injectable drug form prepared by dissolving of the compound in sterile water for injections or in any pharmaceutically acceptable solvent, e.g., to form a resultant concentration of 0.01-3.0%. As yet another example, for an in vitro use in experimental settings, the composition may be dissolved in solvents acceptable for performance of corresponding experiments such as culture media, isotonic saline to solutions, glucose solutions and the like.

For instance, using a concentration of oxidized glutathione (present as a sodium salt) in solution (10.0%, 100 mg/ml) in water for injections (or in normal saline), and using a maximum tolerable volume administered to mice intra-peritoneally (IP, 2.0 ml), intravenously (IV, 0.5 ml), and intramuscularly (1M, 0.05 ml), dosage levels of the composition have reached about 5000 mg/kg (IP), about 1350 mg/kg (IV), or about 135 mg/kg (IM), i.e. 1000, 270, and 27 times.

As mentioned, certain aspects of the present invention provide methods of administering any composition of the present invention to a subject. When administered, the compositions of the invention are applied in a therapeutically effective, pharmaceutically acceptable amount as a pharmaceutically acceptable formulation. As used herein, the term “pharmaceutically acceptable” is given its ordinary meaning. Pharmaceutically acceptable compositions are generally compatible with other materials of the formulation and are not generally deleterious to the subject. Any of the compositions of the present invention may be administered to the subject in a therapeutically effective dose. A “therapeutically effective” or an “effective” as used herein means that amount necessary to delay the onset of, inhibit the progression of, halt altogether the onset or progression of, diagnose a particular condition being treated, or otherwise achieve a medically desirable result. When administered to a subject, effective amounts will depend on the particular condition being treated and the desired outcome. A therapeutically effective dose may be determined by those of ordinary skill in the art, for instance, employing factors such as those further described below and using no more than routine experimentation.

In administering the compositions of the invention to a subject, dosing amounts, dosing schedules, routes of administration, and the like may be selected so as to affect known activities of these compositions. Dosages may be estimated based on the results of experimental models, optionally in combination with the results of assays of compositions of the present invention. Dosage may be adjusted appropriately to achieve desired drug levels, local or systemic, depending upon the mode of administration. The doses may be given in one or several administrations per day.

In the event that the response of a particular subject is insufficient at such doses, even higher doses (or effectively higher doses by a different, more localized delivery to route) may be employed to the extent that subject tolerance permits. Multiple doses per day are also contemplated in some cases to achieve appropriate systemic levels of the composition within the subject or within the active site of the subject.

The dose of the composition to the subject may be such that a therapeutically effective amount of the composition reaches the active site of the composition within the subject. The dosage may be given in some cases at the maximum amount while avoiding or minimizing any potentially detrimental side effects within the subject. The dosage of the composition that is actually administered is dependent upon factors such as the final concentration desired at the active site, the method of administration to the subject, the efficacy of the composition, the longevity of the composition within the subject, the timing of administration, the effect of concurrent treatments (e.g., as in a cocktail), etc. The dose delivered may also depend on conditions associated with the subject, and can vary from subject to subject in some cases. For example, the age, sex, weight, size, environment, physical conditions, or current state of health of the subject may also influence the dose required and/or the concentration of the composition at the active site. Variations in dosing may occur between different individuals or even within the same individual on different days. It may be preferred that a maximum dose be used, that is, the highest safe dose according to sound medical judgment. Preferably, the dosage form is such that it does not substantially deleteriously affect the subject.

Administration of a composition of the invention may be accomplished by any medically acceptable method which allows the composition to reach its target. The particular mode selected will depend of course, upon factors such as those previously described, for example, the particular composition, the severity of the state of the subject being treated, the dosage required for therapeutic efficacy, etc. As used herein, a “medically acceptable” mode of treatment is a mode able to produce effective levels of the composition within the subject without causing clinically unacceptable adverse effects.

Any medically acceptable method may be used to administer the composition to the subject. The administration may be localized (i.e., to a particular region, physiological system, tissue, organ, or cell type) or systemic, depending on the condition to be treated. For example, the composition may be administered orally, vaginally, rectally, buccally, pulmonary, topically, nasally, transdermally, through parenteral to injection or implantation, via surgical administration, or any other method of administration where access to the target by the composition of the invention is achieved. Examples of parenteral modalities that can be used with the invention include intravenous, intradermal, subcutaneous, intracavity, intramuscular, intraperitoneal, epidural, or intrathecal. Examples of implantation modalities include any implantable or injectable drug delivery system. Oral administration may be preferred in some embodiments because of the convenience to the subject as well as the dosing schedule. Compositions suitable for oral administration may be presented as discrete units such as hard or soft capsules, pills, cachettes, tablets, troches, or lozenges, each containing a predetermined amount of the active compound. Other oral compositions suitable for use with the invention include solutions or suspensions in aqueous or non-aqueous liquids such as a syrup, an elixir, or an emulsion.

In certain embodiments of the invention, the administration of the composition of the invention may be designed so as to result in sequential exposures to the composition over a certain time period, for example, hours, days, weeks, months or years. This may be accomplished, for example, by repeated administrations of a composition of the invention by one of the methods described above, or by a sustained or controlled release delivery system in which the composition is delivered over a prolonged period without repeated administrations. Administration of the composition using such a delivery system may be, for example, by oral dosage forms, bolus injections, transdermal patches or subcutaneous implants. Maintaining a substantially constant concentration of the composition may be preferred in some cases.

Other delivery systems suitable for use with the present invention include time-release, delayed release, sustained release, or controlled release delivery systems. Such systems may avoid repeated administrations of the composition in many cases, increasing convenience to the subject. Many types of release delivery systems are available and known to those of ordinary skill in the art. They include, for example, polymer-based systems such as polylactic and/or polyglycolic acids, polyanhydrides, polycaprolactones and/or combinations of these; nonpolymer systems that are lipid-based including sterols such as cholesterol, cholesterol esters, and fatty acids or neutral fats such as mono-, di- and triglycerides; hydrogel release systems; liposome-based systems; to phospholipid based-systems; silastic systems; peptide based systems; wax coatings; compressed tablets using conventional binders and excipients; or partially fused implants. Specific examples include, but are not limited to, erosional systems in which the composition is contained in a form within a matrix (for example, as described in U.S. Pat. Nos. 4,452,775, 4,675,189, and 5,736,152), or diffusional systems in which an active component controls the release rate (for example, as described in U.S. Pat. Nos. 3,854,480, 5,133,974 and 5,407,686). The formulation may be as, for example, microspheres, hydrogels, polymeric reservoirs, cholesterol matrices, or polymeric systems. In some embodiments, the system may allow sustained or controlled release of the composition to occur, for example, through control of the diffusion or erosion/degradation rate of the formulation containing the composition. In addition, a pump-based hardware delivery system may be used to deliver one or more embodiments of the invention.

Use of a long-term release implant may be particularly suitable in some embodiments of the invention. “Long-term release,” as used herein, means that the implant containing the composition is constructed and arranged to deliver therapeutically effective levels of the composition for at least 30 or 45 days, and preferably at least 60 or 90 days, or even longer in some cases. Long-term release implants are well known to those of ordinary skill in the art, and include some of the release systems described above.

In certain embodiments of the invention, a composition can be combined with a suitable pharmaceutically acceptable carrier, for example, as incorporated into a liposome, incorporated into a polymer release system, or suspended in a liquid, e.g., in a dissolved form or a colloidal form. In general, pharmaceutically acceptable carriers suitable for use in the invention are well-known to those of ordinary skill in the art. As used herein, a “pharmaceutically acceptable carrier” refers to a non-toxic material that does not significantly interfere with the effectiveness of the biological activity of the active compound(s) to be administered, but is used as a formulation ingredient, for example, to stabilize or protect the active compound(s) within the composition before use. The term “carrier” denotes an organic or inorganic ingredient, which may be natural or synthetic, with which one or more active compounds of the invention are combined to facilitate the application of the composition. The carrier may be co-mingled or otherwise mixed with one or more active compounds of the present invention, and with each other, in a manner such that there is no interaction which would substantially impair the desired pharmaceutical efficacy. The carrier may be either soluble or insoluble, depending on the application. Examples of well-known carriers include glass, polystyrene, polypropylene, polyethylene, dextran, nylon, amylase, natural and modified cellulose, polyacrylamide, agarose and magnetite. The nature of the carrier can be either soluble or insoluble. Those skilled in the art will know of other suitable carriers, or will be able to ascertain such, using only routine experimentation.

In some embodiments, the compositions of the invention include pharmaceutically acceptable carriers with formulation ingredients such as salts, carriers, buffering agents, emulsifiers, diluents, excipients, chelating agents, fillers, drying agents, antioxidants, antimicrobials, preservatives, binding agents, bulking agents, silicas, solubilizers, or stabilizers that may be used with the active compound. For example, if the formulation is a liquid, the carrier may be a solvent, partial solvent, or non-solvent, and may be aqueous or organically based. Examples of suitable formulation ingredients include diluents such as calcium carbonate, sodium carbonate, lactose, kaolin, calcium phosphate, or sodium phosphate; granulating and disintegrating agents such as corn starch or algenic acid; binding agents such as starch, gelatin or acacia; lubricating agents such as magnesium stearate, stearic acid, or talc; time-delay materials such as glycerol monostearate or glycerol distearate; suspending agents such as sodium carboxymethylcellulose, methylcellulose, hydroxypropylmethylcellulose, sodium alginate, polyvinylpyrrolidone; dispersing or wetting agents such as lecithin or other naturally-occurring phosphatides; thickening agents such as cetyl alcohol or beeswax; buffering agents such as acetic acid and salts thereof, citric acid and salts thereof, boric acid and salts thereof, or phosphoric acid and salts thereof; or preservatives such as benzalkonium chloride, chlorobutanol, parabens, or thimerosal. Suitable carrier concentrations can be determined by those of ordinary skill in the art, using no more than routine experimentation. The compositions of the invention may be formulated into preparations in solid, semi-solid, liquid or gaseous forms such as tablets, capsules, elixirs, powders, granules, ointments, solutions, depositories, inhalants or injectables. Those of ordinary skill in the art will know of other suitable formulation ingredients, or will be able to ascertain such, using only routine experimentation.

Preparations include sterile aqueous or nonaqueous solutions, suspensions and emulsions, which can be isotonic with the blood of the subject in certain embodiments. Examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. Aqueous carriers include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents and inert gases and the like. Those of skill in the art can readily determine the various parameters for preparing and formulating the compositions of the invention without resort to undue experimentation.

In some embodiments, the present invention includes the step of bringing a composition of the invention into association or contact with a suitable carrier, which may constitute one or more accessory ingredients. The final compositions may be prepared by any suitable technique, for example, by uniformly and intimately bringing the composition into association with a liquid carrier, a finely divided solid carrier or both, optionally with one or more formulation ingredients as previously described, and then, if necessary, shaping the product.

In some embodiments, the compositions of the present invention may be present as a pharmaceutically acceptable salt. The term “pharmaceutically acceptable salts” includes salts of the composition, prepared in combination with, for example, acids or bases, depending on the particular compounds found within the composition and the treatment modality desired. Pharmaceutically acceptable salts can be prepared as alkaline metal salts, such as lithium, sodium, or potassium salts; or as alkaline earth salts, such as beryllium, magnesium or calcium salts. Examples of suitable bases that may be used to form salts include ammonium, or mineral bases such as sodium hydroxide, lithium hydroxide, potassium hydroxide, calcium hydroxide, magnesium hydroxide, and the like. Examples of suitable acids that may be used to form salts include inorganic or mineral acids such as hydrochloric, hydrobromic, hydroiodic, hydrofluoric, nitric, to carbonic, monohydrogencarbonic, phosphoric, monohydrogenphosphoric, dihydrogenphosphoric, sulfuric, monohydrogensulfuric, phosphorous acids and the like. Other suitable acids include organic acids, for example, acetic, propionic, isobutyric, maleic, malonic, benzoic, succinic, suberic, fumaric, mandelic, phthalic, benzenesulfonic, p-tolylsulfonic, citric, tartaric, methanesulfonic, glucuronic, galacturonic, salicylic, formic, naphthalene-2-sulfonic, and the like. Still other suitable acids include amino acids such as arginate, aspartate, glutamate, and the like.

The present invention also provides any of the above-mentioned compositions in kits, optionally including instructions for use of the composition for the treatment of cancers or other diseases as described herein. The kit can include a description of use of the compositions as discussed herein. The kit also can include instructions for use of a combination of two or more compositions of the invention, or instruction for use of a combination of a composition of the invention and one or more other compounds indicated for treatment of cancers or other diseases as described herein. Instructions also may be provided for administering the composition by any suitable technique as previously described, for example, orally, intravenously, pump or implantable delivery device, or via another known route of drug delivery.

“Instructions” can define a component of promotion, and typically involve written instructions on or associated with packaging of compositions of the invention. Instructions also can include any oral or electronic instructions provided in any manner The “kit” typically defines a package including any one or a combination of the compositions of the invention and the instructions, but can also include the composition of the invention and instructions of any form that are provided in connection with the composition in a manner such that a clinical professional will clearly recognize that the instructions are to be associated with the specific composition.

The kits described herein may also contain one or more containers, which may contain the inventive composition and other ingredients as previously described. The kits also may contain instructions for mixing, diluting, and/or administrating the compositions of the invention in some cases. The kits also can include other containers with one or more solvents, surfactants, preservative and/or diluents (e.g., normal saline (0.9% NaCl), or 5% dextrose) as well as containers for mixing, diluting or administering the components in a sample or to a subject in need of such treatment.

The compositions of the kit may be provided as any suitable form, for example, as liquid solutions or as dried powders. When the composition provided is a dry powder, the composition may be reconstituted by the addition of a suitable solvent, which may also be provided. In embodiments where liquid forms of the composition are used, the liquid form may be concentrated or ready to use. The solvent will depend on the compound and the mode of use or administration. Suitable solvents for drug compositions are well known, for example as previously described, and are available in the literature. The solvent will depend on the compound and the mode of use or administration.

Each of the following documents is incorporated herein by reference in its entirety: U.S. Pat. No. 6,165,979; U.S. Pat. No. 6,251,857; U.S. Pat. No. 6,492,329; U.S. Pat. No. 6,312,734; U.S. Pat. No. 7,169,412; U.S. Pat. No. 7,371,411; Int. Pat. Apl. Pub. No. WO 97/21443; Int. Pat. Apl. Pub. No. WO 97/21444; Int. Pat. Apl. Pub. No. WO 00/31120; and Int. Pat. Apl. Pub. No. WO 97/21444.

Also incorporated herein by reference in its entirety is U.S. Provisional Patent Application Ser. No. 61/241,808, filed Sep. 11, 2009, entitled “Treatment of Metastatic Tumors and Other Conditions,” by Pazoles, et al.

The following examples are intended to illustrate certain embodiments of the present invention, but do not exemplify the full scope of the invention.

### EXAMPLE 1

This example illustrates the use of a composition of the invention for the treatment of tumors. The composition in this example was a 1000:1 molar ratio of oxidized glutathione (GSSG) to cisplatin (Pt(NH₃)₂Cl₂), and may be referred to as “NOV-002.” As discussed above, however, other compositions could be used instead, e.g., any composition comprising a metal material such as platinum material, palladium material, or the like.

This example illustrates that this composition dose-responsively inhibited tumor cell invasion in 5 of 8 cell lines studied and inhibited migration in 2 of the 8. In both regards, the IC50 of the composition was approximately 30 micromolar, and close to 100% inhibition was achievable. The composition also decreased the levels of phosphorylated, activated forms of signaling proteins (e.g., ErbB2, PIK3, RhoA, and AKT) known to regulate these in vitro processes, as well as tumor cell metastasis in vivo. Without being bound by any theory, these preliminary results are consistent with the hypothesis that redox modulation by an oxidized glutathione-based compound and the resulting alterations in cytoskeletal and cell surface proteins may inhibit processes essential for tumor metastasis. Such anti-metastatic activity could contribute to the clinical profile of the inventive compositions in cancer patients treated with standard chemotherapy which includes increased efficacy (survival, tumor response).

As mentioned, the composition used in this example included a formulation of oxidized glutathione, or GSSG, although other compositions comprising oxidized glutathione-based compounds could also be used. It is believed that such a composition applied to the myeloid lineage cell line HL-60 may induce oxidative signals and protein modification (glutathionylation) at the cell surface and/or intracellularly. One such protein modified by the composition is actin, glutathionylation of which can regulate cytoskeletal structure and function in some cases. In addition, a composition comprising an oxidized glutathione-based compound may also glutathionylate cell surface proteins, possibly including protein disulfide isomerase (PDI), whose activity it inhibits. Finally, both the actin polymerization cycle and surface PDI activity may be important regulators of the migration, invasiveness and metastasis of tumor cells in vitro and in vivo. Thus, the experiments shown in this example were designed to evaluate the effects of the composition described above in in vitro models of tumor cell migration and invasiveness.

Human tumor cell lines studied to date include non-small cell lung cancer (A549), breast cancer (MDA-MB-436, MDA-MB-435, MDA-MB-231), colon cancer (HCT116, HCT15, Colo205), and prostate (PC3). Tumor cell migration and invasion assays were carried out using well-known methods briefly described below.

For the cell migration assay, a cell migration/invasion chamber was employed that included upper and lower compartments separated by a microporous polycarbonate membrane (8 micron pore diameter; see diagram below). See FIG. 1. Cells were able to enter, but not easily cross this membrane. Culture medium containing fetal bovine serum (FBS) was placed in the lower compartment of the chamber. The FBS provides chemoattractant stimuli such as integrin ligands to induce movement of tumor cells from the upper compartment to the lower chamber. 2×10⁵ tumor cells were incubated with DMSO (as a control) or with various concentrations of the compositions described above. After 30 minutes, the cells were transferred to the upper compartment of the cell migration/invasion chamber. Approximately 16 hours later, the upper chamber was removed, the top surface of the membrane wiped clean, and the membrane stained with crystal violet to stain penetrating cells. Cells on the bottom surface of the membrane were quantified by microscopic observation (4 fields/membrane).

For the cell invasion assay, the microporous membrane was overlaid with Matrigel™, which resembles the extracellular matrix through which tumor cells must “invade” in order to metastasize. The cell migration/invasion chamber and methods used were otherwise identical.

Each condition was performed in triplicate. Data are displayed as mean±SE.

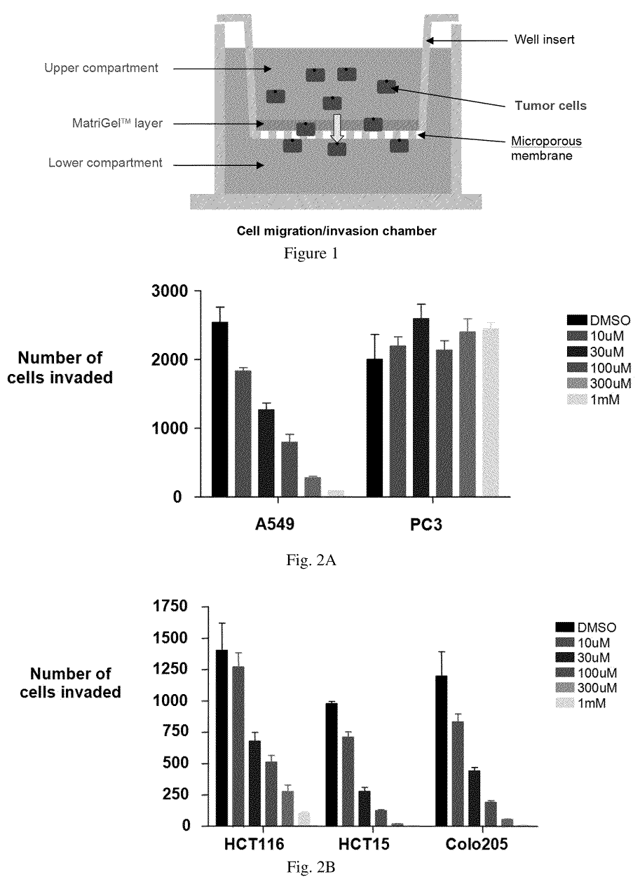

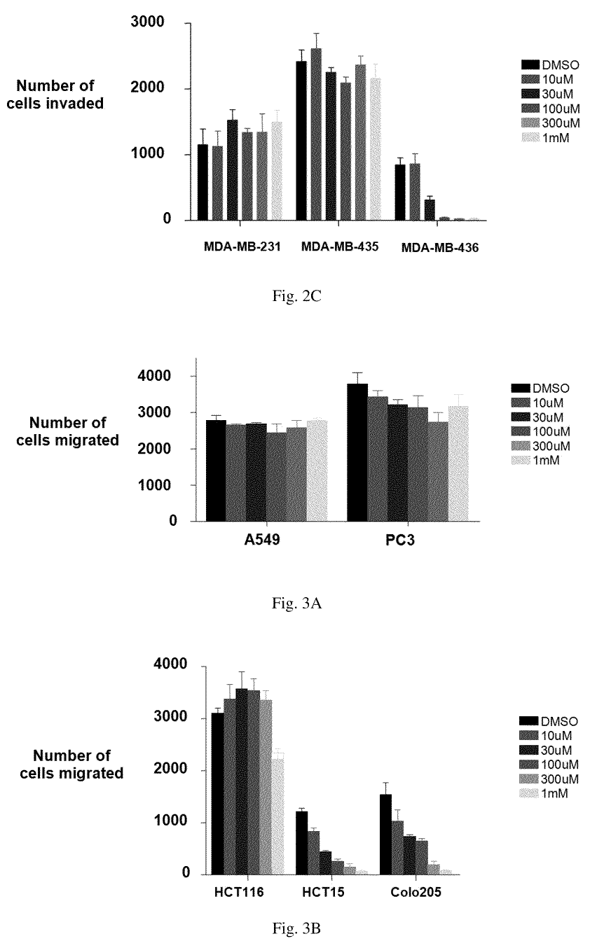

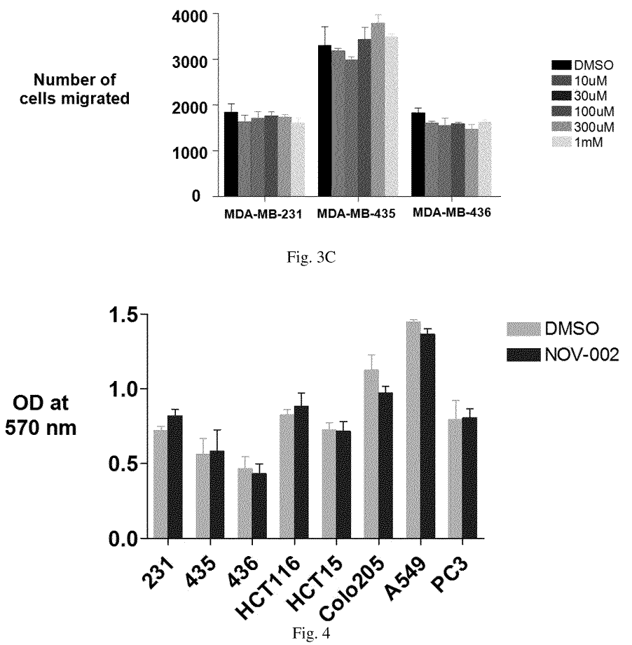

FIGS. 2A-2C shows the results for the tumor cell invasion assay. Control levels of invasion (darkest bars; DMSO) were in agreement with prior studies, with the cell lines displaying varying degrees of invasion activity. In contrast, the composition described above dose-responsively inhibited invasion of 5 of the 8 tumor cell lines studied (A549, MDA-MB-436, HCT116, HCT15 and Colo205). In each case, 50% inhibition was seen at approximately 30 micromolar, and invasion was essentially totally inhibited at 1 mM. Similarly, FIGS. 3A-3C illustrates results for the tumor cell migration assay. As for migration, control levels of migration (black bars; DMSO) were in agreement with previous studies, with the cell lines displaying varying degrees of migration activity. In contrast, the composition described above dose-responsively inhibited migration of 2 of the 8 tumor cell lines studied (HCT15 and Colo205). In both cases, 50% inhibition was seen at approximately 30 micromolar NOV-002, and invasion was essentially totally inhibited at 1 mM. FIG. 4 shows that 1 mM of the composition described above (i.e., the highest concentration tested in the invasion and migration assays used in this example) was not directly toxic to any of the tumor cell lines studied even after 72 hours of culture (MTT viability assay).

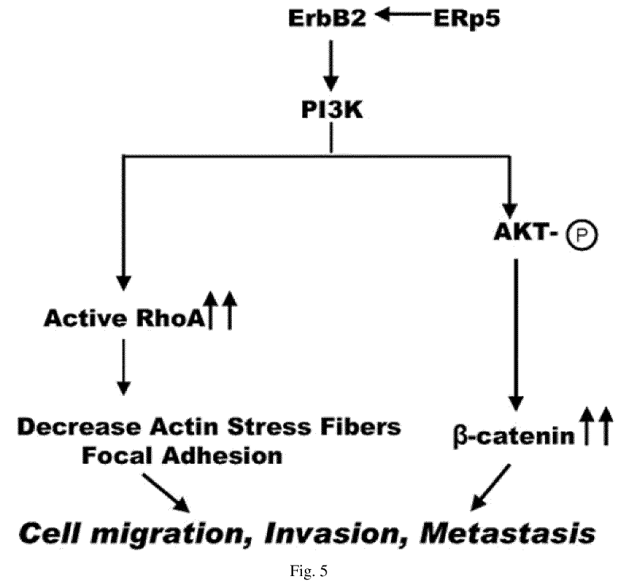

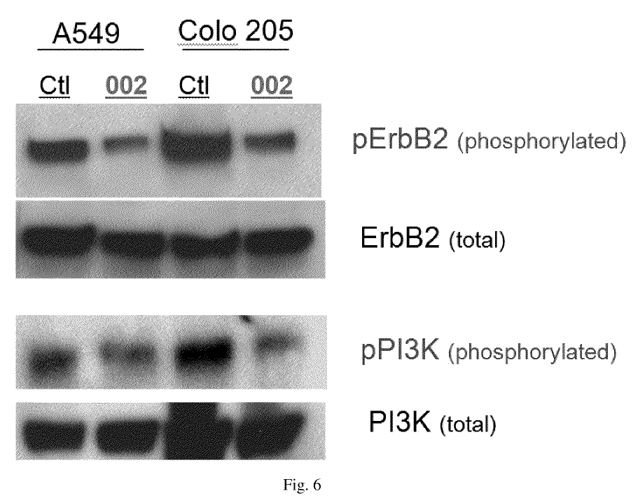

The cell signaling pathway that has been implicated in regulation of tumor cell migration, invasion and metastasis is depicted in FIG. 5. The effect of the composition described above on these pathways was examined in another experiment by incubating two human tumor cell lines which displayed invasion sensitivity to the composition, A549 and Colo 205. After 24 hrs of incubation in the presence or absence of 1 mM of the composition, the total and phosphorylated levels of ErbB2 and PI3K were measured. FIG. 6 shows that the composition as described above decreased the amount of the to activated, phosphorylated forms of these proteins (pErbB2 and pPI3K) without affecting the total amount of these proteins (ErbB2 and PI3K). Thus, the observed inhibition of invasion by these tumor cell lines may be due to decreased activation of the signaling pathway that controls this process. Indeed, the most upstream point of this pathway is ERp5, a member of the PDI protein family, which may represent a target for the composition studied here since, as indicated above, it may inhibit PDI activity in a cell-free system.

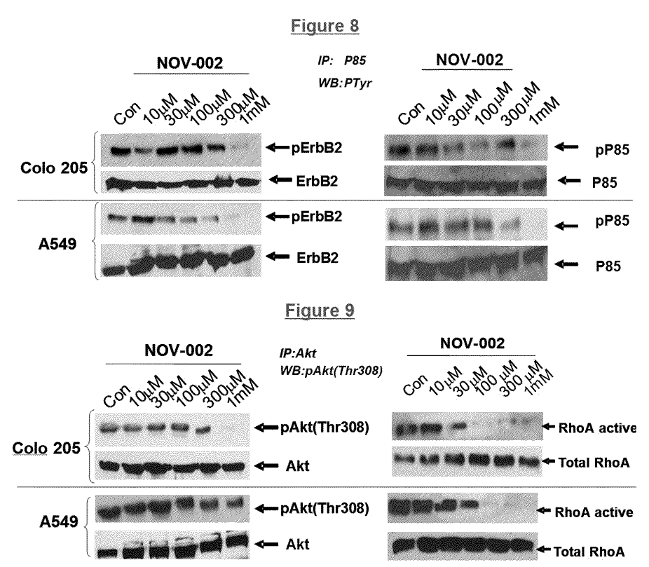

FIG. 8 illustrates that the composition of Example 1 suppresses ErbB2/PI3K pathway activity. In particular, this composition reduces the expression of phosphorylated ErbB2 and PI3K, although it did not appear to have a significant effect on the total protein expression of these two molecules. Similarly, FIG. 9 illustrates that the composition of Example 1 is able to reduce Akt and RhoA expression. It appeared to reduce the expression of the active from of Akt and RhoA, but did not appear to have a significant effect on the total protein expression of these two molecules. Akt and RhoA are downstream molecules in the ErbB2/PI3K pathway (see FIG. 5).

### EXAMPLE 2

Tumor cell invasion (through extracellular matrix) and migration are known to depend, in part, on cytoskeletal rearrangements mediated by the actin polymerization cycle and on the action of cell surface proteins regulated by the enzyme protein disulfide isomerase (PDI). Both actin and PDI appear to be targets of structural and functional modification by the composition used in Example 1 via the process of protein S-glutathionylation. (Townsend, et al., Cancer Research, 2008; 68:2870-2877, the contents of which are incorporated by reference in their entirety.) This led to the hypothesis that this composition may be capable of inhibiting tumor cell invasion/migration/metastasis.

As discussed below, the composition used in Example 1 dose-responsively inhibited invasion in cell lines derived from NSCLC (A549), colon (HCT116, HCT15, Colo205) and breast (MDA-MB-436) tumor tissue Inhibition was about 50% at 30 micromolar and reached 100% at the highest dose tested (1 mM). However, this composition did not appear to be as effective on the invasion in a prostate tumor cell line (PC3) and in two other breast tumor lines (MDA-MB-231, MDA-MB-435). These results suggest that the composition may suppress tumor metastasis under some to conditions. These data also suggest that the regulation of tumor cell invasion may differ between cell lines.

Without being bound by any theory, an understanding of the basis of such differences in sensitivity of tumor cell lines to the composition used in Example 1 may provide important clues as to the molecular mechanism of the composition's actions in this system, perhaps ultimately extending to indications of which cancer patients may receive the greatest clinical benefit. In this regard, the effects of the composition on cells may, at least in part, depend upon the activity of cell surface gamma-glutamyltransferase (GGT). The action of this enzyme on GSSG can lead to generation of hydrogen peroxide which could represent one source of oxidative signaling subsequent to exposure of cells to the composition. The potential relevance of GGT to inhibition of tumor cell invasion is suggested by the fact that, of the 8 tumor cell lines tested, there are literature reports for two regarding GGT expression—A549 that expresses GGT and is sensitive to NOV-002 and PC3 that does not express GGT and is not sensitive to the composition. Thus, cell surface GGT may be a predictive marker for inhibition of tumor cell invasion by the composition. Similarly, in the tumor cell migration assay, the composition used in Example 1 was similarly active against two of the colon cancer cell lines—HCT15 and Colo205.

The composition used here was capable of inhibiting tumor cell invasion and not migration in some cell lines. Cell migration is known to be necessary, but not sufficient, for cell invasion. It is thus appropriate that both cell lines in which the composition appeared to inhibit migration inhibited invasion was also blocked (HCT15 and Colo205); and that no invasion-insensitive lines were inhibited in their migration by the composition. These data also suggest that, in some tumors, different pathways may mediate migration and invasion while in others it is possible that some overlap of pathways for the two processes may exist.

Finally, consistent with its ability to inhibit tumor cell migration and invasion, the composition used in Example 1 was shown to decrease the levels of phosphorylated, activated forms of signaling proteins (ErbB2, PIK3, RhoA, and AKT) known to regulate these in vitro processes as well as tumor cell metastasis in vivo. As activation of these signaling proteins is dependent upon the activity of a PDI-family protein (ERp5), this effect of this composition may be related to its ability to inhibit PDI, a known redox-regulated cell surface protein.

In conclusion, these results with tumor cells are consistent with the hypothesis that redox modulation by the composition used in Example 1 and the resulting alterations in cytoskeletal and cell surface proteins such as PDI inhibit processes important or even essential for tumor metastasis. Such anti-metastatic activity is surprising, and—in addition to the known hematopoietic, immune stimulating, and chemosensitizing properties—could contribute to the clinical profile of this composition in cancer patients which includes increased efficacy (survival, tumor response). Also, in some cases, levels of activated phosphorylated forms of the signaling kinases were inhibited, but total levels of these proteins were not. In addition, this composition may also prove to be a novel and useful tool for identifying, dissecting and regulating cellular pathways involved in the metastatic process.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not to necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method of inhibiting metastasis of tumor cells, comprising:
administering, to a subject diagnosed as having a metastatic tumor, a composition comprising an oxidized glutathione-based compound, wherein the compound inhibits metastasis of the tumor cells.

2. The method of claim 1, wherein the metastatic tumor comprises cells exhibiting migration and/or invasion of at least about 500 migrating cells, as determined by a transwell Matrigel migration/invasion assay using 100 microliters of cells at a density of 10⁶ cells/ml in a donor chamber and 600 microliters of culture media in a receiving chamber separated by a 1 mg/ml Matrigel layer having a thickness of about 100 mm, after incubation at 37° C. for 24 hours.

3. The method of claim 1, comprising administering the composition orally to the subject.

4. The method of claim 1, wherein the composition is administered as a solution form selected from the group consisting of inhalation solutions, local instillations, eye drops, intranasal introductions, ointment for epicutaneous applications, intravenous solutions, injection solutions, and suppositories.

5. The method of claim 1, wherein the oxidized glutathione-based compound is formed from two monomers, each monomer comprising a glutamic acid bonded to a cysteine bonded to a glycine, the two monomers being linked through a disulfide bond bridging the sulfur atoms of each respective cysteine of each monomer.

6. The method of claim 1, wherein the oxidized glutathione-based compound is selected from the group consisting of the formula: 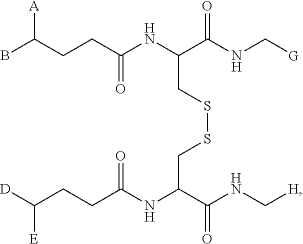
and salts of said formula, wherein A, B, D, E, G and H can be the same or different and each is selected from the group consisting of an organic unit and salts of the organic unit.

7. The method of claim 6, wherein A, B, D, E, G and H can be the same or different and each includes a unit selected from the group consisting of amine groups, carboxyl groups and amides.

8. The method of claim 7, wherein the oxidized glutathione-based compound is oxidized glutathione and both A and E are —CO₂H, both B and D are —NH₂ and both G and H are —CO₂M, M being a counterion.

9. The method of claim 1, wherein the composition further comprises a metal material.

10. The method of claim 9, wherein the metal material comprises platinum material.

11. The method of claim 9, wherein the metal material further comprises cis-platin.

12. The method of claim 1, wherein the oxidized glutathione-based compound comprises GSSG.

13. The method of claim 1, wherein the composition comprises GSSG and cis-platin.

14. The method of claim 13, wherein the molar equivalent ratio of GSSG to cis-platin is between about 3000:1 and about 1:1.

15. The method of claim 13, wherein the molar equivalent ratio of GSSG to cis-platin is between about 1500:1 and about 500:1.

16. The method of claim 1, wherein the composition further comprises an extender of the half life of the oxidized glutathione-based compound.

17. The method of claim 1, wherein the composition further comprises inosine.

18. The method of claim 1, wherein the composition comprises GSSG and inosine.

19. The method of claim 18, wherein the molar equivalent ratio of GSSG to inosine is between about 5:1 and about 1:5.

20. Use of a composition in the preparation of a medicament for treatment of a metastatic tumor, the composition comprising a compound comprising an oxidized glutathione-based compound.
